(19) United States
(12) Plant Patent Application Publication
(10) Pub. No.: US 2002/0073466 P1
(43) Pub. Date: Jun. 13, 2002

(54) Variety of geranium plant named 'Penber'

(76) Inventor: Wilhelmus Johannes Maria Ibes, Oosterhout (NL)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,020

(22) Filed: Dec. 8, 2000

(51) Int. Cl. A01H 5/00

(52) U.S. Cl. PLT/330

(57) ABSTRACT

A new and distinct zonal geranium plant with intense pink colored flowers above two-colored medium green foliage. The plant flower is suitable for pot planting.

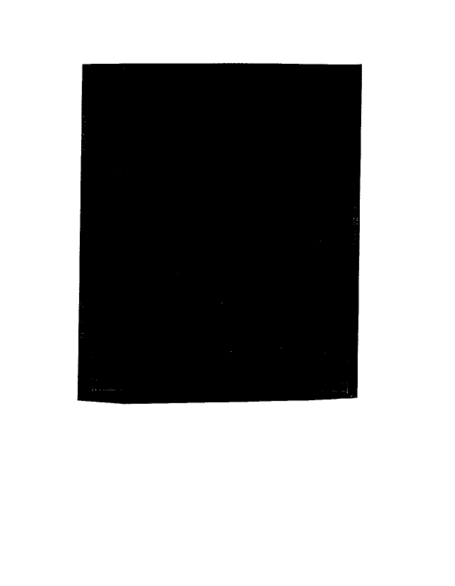

### BACKGROUND OF THE INVENTION

The present invention comprises a new and distinct cultivar of Pelargonium×hortorum known by the varietal name ‘Penber’. The new variety was discovered in 1996 in a selective breeding program in Oosterhout, The Netherlands. The new variety is a mutation of ‘Melody’ (PEL 676). The new variety exhibits the flower color of its parent, but differs in its flower shape, leaf color and leaf shape. The new variety was first asexually reproduced by cuttings in 1997. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to the R.H.S. Colour Chart of The Royal Horticultural Society of London.

Plant:

Classification:

Botanical.—Pelargonium×hortorum.

Commercial.—Zonal geranium.

Form.—Erect.

Height from media surface to top of foliage.—22-25 cm.

Height from media surface to top of umbel.—30 cm.

Growth.—Erect, very compact, well-branched.

Strength.—Does not require artificial support.

Response time.—4 weeks.

Leaves:

Size.—6-8 cm long.

Shape.—Cordate, ondulated.

Margin.—Partially toothed.

Texture.—Smooth.

Color.—Green Group 138A with edges of Green-White Group 157D.

Ribs and veins:

Color.—Darker than leaf surface.

Petioles:

Color.—Medium green.

Length.—4 cm.

Stem/branches:

Color.—Medium Green.

Internode length.—1-2 cm.

The bud:

Shape when just showing color:

Overall.—Semi-spherical.

Individual bud.—Narrow; elliptical.

Color.—Green Group 143C.

Size when just showing color:

Umbel.—5 cm across.

Individual bud.—1 cm long; 0.5-0.8 cm wide.

Inflorescence:

Blooming habit.—Semi-double.

Size of fully open bloom.—2-2.5 cm across.

Borne.—Flowers on umbel; umbel on pedicel; pedicel on peduncle.

Flowers:

Form.—Zygomorph.

Number of petals.—7-10.

Size.—1 cm across; 1-1.5 cm long.

Petals.—

Color:

Upper surface.—Red-Purple Group 68C.

Lower surface.—Red-Purple Groups 67C-D.

Texture and appearance.—Velvety.

Petaloids:

Quantity.—7.

Shape.—Flakes; ondulated.

Color:

Upper surface.—Red-Purple Group 68B.

Lower surface.—Red-Purple Groups 67 C-D.

Pedicel:

Length.—2 cm.

Color.—Medium green.

Peduncle:

Length.—5-8 cm.

Color.—Medium green.

Disease/pest resistance.—No unusual susceptibility to diseases or pests has been noted to date.

Lasting Quality.—Long-lasting; 3 weeks per umbel.

Reproductive organs:

Stamens:

Anthers.—3 mm long.

Filaments:

Length.—0.5 cm maximum.

Color.—Pink.

Pollen.—Yellow.

Pistils:

Number.—Five.

Length.—0.3-0.4 cm.

Stigma.—Pink.

Style.—0.8 cm long.

Ovaries:

Color.—Greenish.

Length.—0.4 cm.

Fruit.—Absent.

### GENERAL CHARACTERISTICS

1. Very compact.

2. Very decorative as pot plant.

3. Chimeric plant with two-colored foliage.

I claim:

1. A new and distinct variety of geranium plant substantially as shown and described.
